SEQUENCE LISTING (36 sequences)

SEQ ID NO: 1 - DNA, 18 bp, Artificial Sequence
  Description of Artificial Sequence Synthetic primer
ctgggaaggatcagcctc

SEQ ID NO: 2 - DNA, 45 bp, Artificial Sequence
  Description of Artificial Sequence Synthetic primer
gtttgtaatatactgctctctcctgccttgcattttctgcttgac

SEQ ID NO: 3 - DNA, 20 bp, Artificial Sequence
  Description of Artificial Sequence Synthetic primer
aaatgaagatgttggagacg

SEQ ID NO: 4 - DNA, 49 bp, Artificial Sequence
  Description of Artificial Sequence Synthetic primer
caccaaaaaatatacttatttacgcctgcaggtatgtattcatagactc

SEQ ID NO: 5 - DNA, 22 bp, Artificial Sequence
  Description of Artificial Sequence Synthetic primer
gtaccagattcctctctcttct

SEQ ID NO: 6 - DNA, 14 bp, Artificial Sequence
  Description of Artificial Sequence Synthetic probe
gtgctgcaggtgcg

SEQ ID NO: 7 - DNA, 26 bp, Artificial Sequence
  Description of Artificial Sequence Synthetic probe
ttgaacctcgaacaattgaagatttt

SEQ ID NO: 8 - DNA, 27 bp, Artificial Sequence
  Description of Artificial Sequence Synthetic probe
ttgaacctcaaacaattgaagattttg

SEQ ID NO: 9 - DNA, 25 bp, Artificial Sequence
  Description of Artificial Sequence Synthetic primer
gagcttggcatattgtatctatacc

SEQ ID NO: 10 - DNA, 59 bp, Artificial Sequence
  Description of Artificial Sequence Synthetic primer
tcactttccataaaagcaaggtttttaagtaactcttagatatgcaataattttcccac

SEQ ID NO: 11 - DNA, 24 bp, Artificial Sequence
  Description of Artificial Sequence Synthetic primer
ctagtcaatgaatcacaaatacgc

SEQ ID NO: 12 - DNA, 54 bp, Artificial Sequence
  Description of Artificial Sequence Synthetic primer
agaaagtaaaagaacaccaagaatcgatgtaacattttaccttctccattttga

SEQ ID NO: 13 - DNA, 19 bp, Artificial Sequence
  Description of Artificial Sequence Synthetic primer
catcaacaaccctcgggac

SEQ ID NO: 14 - DNA, 17 bp, Artificial Sequence
  Description of Artificial Sequence Synthetic probe
gggttcctgggaaataa

SEQ ID NO: 15 - DNA, 21 bp, Artificial Sequence
  Description of Artificial Sequence Synthetic probe
tatgggttcctgggaaataat

SEQ ID NO: 16 - DNA, 23 bp, Artificial Sequence
  Description of Artificial Sequence Synthetic probe
ttatgggttcctgggaaataatc

SEQ ID NO: 17 - DNA, 24 bp, Artificial Sequence
  Description of Artificial Sequence Synthetic probe
ttatgggttcctgggaaataatca

SEQ ID NO: 18 - DNA, 30 bp, Artificial Sequence
  Description of Artificial Sequence Synthetic probe
ttgttatgggttcctgggaaataatcaatg

SEQ ID NO: 19 - DNA, 18 bp, Artificial Sequence
  Description of Artificial Sequence Synthetic primer
gtctcctgccctgacagc

SEQ ID NO: 20 - DNA, 39 bp, Artificial Sequence
  Description of Artificial Sequence Synthetic primer
cagtggtttcttcatcccgcaggcacatcttgttccctc

SEQ ID NO: 21 - DNA, 19 bp, Artificial Sequence
  Description of Artificial Sequence Synthetic primer
actccttggtgtgctcctc

SEQ ID NO: 22 - DNA, 42 bp, Artificial Sequence
  Description of Artificial Sequence Synthetic primer
ggaaggctcagtataaatagcagtgctgtagctgagctgcgg

SEQ ID NO: 23 - DNA, 24 bp, Artificial Sequence
  Description of Artificial Sequence Synthetic primer
gtcatttatcccagttgtgcaacc

SEQ ID NO: 24 - DNA, 24 bp, Artificial Sequence
  Description of Artificial Sequence Synthetic primer
gaggctatttttgatcacattgta

SEQ ID NO: 25 - DNA, 41 bp, Artificial Sequence
  Description of Artificial Sequence Synthetic primer
gaaaaccgattgtggtcagagggtgtctccaggtcaatcaa

SEQ ID NO: 26 - DNA, 18 bp, Artificial Sequence
  Description of Artificial Sequence Synthetic primer
ggctgccacatctgggag

SEQ ID NO: 27 - DNA, 37 bp, Artificial Sequence
  Description of Artificial Sequence Synthetic primer
catggttcaccttctcctgagcttccagacccagcat

SEQ ID NO: 28 - DNA, 16 bp, Artificial Sequence
  Description of Artificial Sequence Synthetic primer
cccagtacagaagttg

SEQ ID NO: 29 - DNA, 17 bp, Artificial Sequence
  Description of Artificial Sequence Synthetic primer
gtgggcttcatcctcac

SEQ ID NO: 30 - DNA, 46 bp, Artificial Sequence
  Description of Artificial Sequence Synthetic primer
agcacttcttcaacctcttcctctaaagacaatacagatctggtcg

SEQ ID NO: 31 - DNA, 23 bp, Artificial Sequence
  Description of Artificial Sequence Synthetic primer
tgataattagtgagttgggtgat

SEQ ID NO: 32 - DNA, 43 bp, Artificial Sequence
  Description of Artificial Sequence Synthetic primer
ggggagaaatctcgtgcccaagggtttattttgttccttattc

SEQ ID NO: 33 - DNA, 22 bp, Artificial Sequence
  Description of Artificial Sequence Synthetic primer
agtgagagttttaaactcgacc

SEQ ID NO: 34 - DNA, 17 bp, Artificial Sequence
  Description of Artificial Sequence Synthetic probe
ccaccgttccctggcag

SEQ ID NO: 35 - DNA, 17 bp, Artificial Sequence
  Description of Artificial Sequence Synthetic probe
aggtgaccactgacggc

SEQ ID NO: 36 - DNA, 23 bp, Artificial Sequence
  Description of Artificial Sequence Synthetic probe
cctggtgatggatcccttactat

(19) United States
(12) Patent Application Publication
(10) Pub. No.: US 2009/0275028 A1
(43) Pub. Date: Nov. 5, 2009

(54) METHOD OF DETECTING TARGET NUCLEIC ACID

(76) Inventors: Naoko Nakamura, Kawasaki-shi (JP); Koji Hashimoto, Atsugi-shi (JP); Nobuhiro Gemma, Yokohama-shi (JP)

Correspondence Address: OBLON, SPIVAK, MCCLELLAND MAIER & NEUSTADT, L.L.P., 1940 DUKE STREET, ALEXANDRIA, VA, 22314, US

(21) Appl. No.: 12/329,898

(22) Filed: Dec. 8, 2008

(30) Foreign Application Priority Data
Apr. 30, 2008 (JP) 2008-119297

(51) Int. Cl. C12Q 1/68

(52) U.S. Cl. 435/6

(57) ABSTRACT

The present invention provides a method of detecting a target nucleic acid which includes a step of examining whether a washing step has been normally conducted. In an aspect of the invention, a monitoring nucleic acid probe to monitor the washing level is used. The probe shows a change in signal intensity by washing at a washing temperature changed in the optimum temperature range for washing the target nucleic acid and in a temperature range in the vicinity of the optimum temperature range for washing.

### CROSS-REFERENCE TO RELATED APPLICATIONS

This application is based upon and claims the benefit of priority from prior Japanese Patent Application No. 2008-119297, filed Apr. 30, 2008, the entire contents of which are incorporated herein by reference.

### BACKGROUND OF THE INVENTION

1. Field of the Invention

The present invention relates to a method of detecting a target nucleic acid which includes a step of examining an abnormality in a washing step.

2. Description of the Related Art

For detection of a target nucleic acid, a microarray can be used. A nucleic acid probe is immobilized on the microarray. A target nucleic acid is hybridized with this immobilized nucleic acid probe. Generally, a negative control probe and a positive control probe are used in detection of a target nucleic acid with a microarray. The negative control probe is used for determining a background value (standard value). The positive control probe is used for examining an abnormality in a series of steps including extraction, amplification and detection of a target nucleic acid or in a part of such steps.

When the signal intensity from the positive control probe is detected at levels not lower than a predetermined value, it is judged that the test was normally conducted. On the other hand, the signal intensity from the positive control probe is not sufficiently detected, it is judged that there was an abnormality in any of the steps (for example, JP-A 2007-506402 (KOKAI)).

However, there may occur unspecific hybridization between a target nucleic acid and a nucleic acid probe. Particularly, a target nucleic acid with single nucleotide polymorphism (SNP) or with insertion or deletion of several bases is liable to unspecific reaction because its wild-type sequence and mutant-type sequence are similar to each other. For example, a wild-type target nucleic acid can be hybridized not only specifically with a probe for wild-type detection but also nonspecifically with a probe for mutant-type detection. Similarly, a mutant-type target nucleic acid can be hybridized not only specifically with a probe for mutant-type detection but also nonspecifically with a probe for wild-type detection. When such unspecific hybrids therebetween are generated, homogeneous ones are erroneously judged as heterogeneous ones.

Similarly, when closely related organisms, microorganisms or viruses are to be detected, unspecific hybrids may be generated to cause erroneous judgment of negative ones as positive ones.

Usually, such unspecific hybrids are removed by washing after hybridization reaction. However, washing may not be normally conducted due to some inconveniences. However, an abnormality in the washing step cannot be found with the positive control described above. Accordingly, there has conventionally been a problem that a sample which was not normally washed is not eliminated, thus causing erroneous judgment.

### BRIEF SUMMARY OF THE INVENTION

The level of washing strength is influenced mainly by temperature and salt concentration during washing. Generally, the washing level is increased under the conditions of a higher temperature and a lower salt concentration. On the other hand, the washing level is decreased under the conditions of a lower temperature and a higher salt concentration. With respect to pH, the washing level is not significantly influenced in the range of pH 5 to 9.

An abnormality in the washing step may occur in washing at a temperature lower than a predetermined temperature, due to inconvenience in temperature control by an apparatus. In this case, the washing level is decreased to make removal of unspecific hybrids insufficient. However, a method of detecting such inconvenience has been nonexistent so far.

Accordingly, it is necessary to develop a method of detecting an abnormality in a washing step, thereby developing an examination method capable of accurately detecting a target nucleic acid even if similar sequences exist.

According to a first aspect of the invention, there is provided a method of using a monitoring nucleic acid probe and a monitoring nucleic acid for monitoring a washing level, to indicate whether washing was conducted normally or not. The monitoring nucleic acid comprises a sequence complementary to the monitoring nucleic acid probe. The monitoring nucleic acid probe is a probe that is highly sensitive under optimum washing conditions for the target nucleic acid. The degree of hybridization of the probe varies due to a slight change in the temperature of a washing fluid. That is, a hybrid formed between the probe and its complementary chain is increased or decreased, thus changing the signal intensity detected from the hybrid. This monitoring nucleic acid probe is immobilized on for example the same substrate which a probe for target nucleic acid detection is immobilized on. Hereafter, the probe for target nucleic acid detection refers to the nucleic acid probe.

In the present invention, the monitoring nucleic acid is hybridized with the monitoring nucleic acid probe and then washed under suitable washing conditions, upon which the signal intensity obtained from the hybrid generated is measured to determine a suitable signal intensity range (optimum signal intensity range for washing). For detecting a target nucleic acid, the target nucleic acid and the monitoring nucleic acid are simultaneously provided to a detection system containing the nucleic acid probe and the monitoring nucleic acid probe. When the signal intensity detected from the monitoring nucleic acid probe is within the optimum signal intensity range for washing, it can be assured that the washing step was normally conducted. On the other hand, when the signal intensity thus detected is outside the optimum signal intensity range for washing, it can be judged that there is an abnormality in the washing step.

According to a second aspect of the invention, there is provided a method of detecting a target nucleic acid, comprising:

preparing the target nucleic acid and a monitoring nucleic acid for monitoring a washing level, the target nucleic acid comprises a target sequence and the monitoring nucleic acid comprises a sequence not to be hybridized with the target sequence or with a sequence complementary to the target sequence,

providing the target nucleic acid and the monitoring nucleic acid to a nucleic acid probe comprising a sequence complementary to the target sequence and a monitoring nucleic acid probe to monitor the washing level, the monitoring nucleic acid probe comprising a sequence complementary to the sequence comprised in the monitoring nucleic acid, thereby hybridizing the target nucleic acid with the nucleic acid probe and hybridizing the monitoring nucleic acid with the monitoring nucleic acid probe,

washing hybrids generated in the above step to remove unspecific hybrids,

measuring the signal intensity from the nucleic acid probe hybridized with the target nucleic acid and the signal intensity from the monitoring nucleic acid probe hybridized with the monitoring nucleic acid, respectively, and

examining whether the washing step has been normally conducted or not;

wherein the monitoring nucleic acid probe shows a change in signal intensity after hybridization with the monitoring nucleic acid and subsequent washing at a washing temperature changed in the optimum temperature range for washing and a vicinity of the range,

an optimum signal intensity range for washing having the upper and lower limits is determined in advance based on the signal intensity obtained from the monitoring nucleic acid probe which has been hybridized with monitoring nucleic acid and subsequent washed in

the optimum temperature range for washing, and the examining step comprises judging that when the signal intensity obtained from the monitoring nucleic acid probe is within the optimum signal intensity range for washing, the washing has been normally conducted, and that when the signal intensity is outside the optimum signal intensity range for washing, there has been an abnormality in the washing step.

Additional objects and advantages of the invention will be set forth in the description which follows, and in part will be obvious from the description, or may be learned by practice of the invention. The objects and advantages of the invention may be realized and obtained by means of the instrumentalities and combinations particularly pointed out hereinafter.

### BRIEF DESCRIPTION OF THE SEVERAL VIEWS OF THE DRAWING

The accompanying drawings, which are incorporated in and constitute a part of the specification, illustrate embodiments of the invention, and together with the general description given above and the detailed description of the embodiments given below, serve to explain the principles of the invention.

FIG. 1 is a schematic diagram showing one example of the probe-immobilized substrate;

FIG. 2 is a schematic diagram showing another example of the probe-immobilized substrate;

FIG. 3A is a graph showing the results in Example 1 (at 44 ° C. and 45° C.);

FIG. 3B is a graph showing the results in Example 1 (at 46° C. and 47° C.);

FIG. 3C is a graph showing the results in Example 1 (at 48° C. and 49° C.);

FIG. 3D is a graph showing the results in Example 1 (at 50° C. and 51° C.);

FIG. 3E is a graph showing the results in Example 1 (at 52° C.);

FIG. 4A is a scatter chart showing the results in Example 1 (G type);

FIG. 4B is a scatter chart showing the results in Example 1 (A type);

FIG. 4C is a scatter chart showing the results in Example 1 (G/A type);

FIG. 5 is a graph showing the results in Example 2;

FIG. 6A is a graph showing the results in Example 3 (SEQ ID NO:34);

FIG. 6B is a graph showing the results in Example 3 (SEQ ID NO:16);

FIG. 6C is a graph showing the results in Example 3 (SEQ ID NO:35);

FIG. 6D is a graph showing the results in Example 3 (SEQ ID NO:36);

FIG. 7A is a graph showing the results in Example 4, washing at 48.5° C. and the washing flued was normally sent;

FIG. 7B is a graph showing the results in Example 4, washing at 44° C. and the washing flued was normally sent; and

FIG. 7C is a graph showing the results in Example 4, washing at 48.5° C. and the washing flued was not sent normally.

### DETAILED DESCRIPTION OF THE INVENTION

The term “nucleic acid” used herein is intended to refer collectively to substances such as DNA, RNA, LNA, S-oligo and methyl phosphonate oligo, a partial structure of which can be expressed in nucleotide structure.

In the present invention, the term “target nucleic acid” means a nucleic acid to be detected by the method of the present invention.

In the present invention, the term “target sequence” means a sequence comprised in a target nucleic acid. The target sequence is used to detect a target nucleic acid.

In the present invention, the term “nucleic acid probe” means a probe for target nucleic acid detection. The nucleic acid probe comprises a sequence complementary to a target sequence. The nucleic acid probe forms a hybrid with a target nucleic acid.

In the present invention, the term “monitoring nucleic acid” means a nucleic aid prepared for monitoring the level of washing strength. The monitoring nucleic acid comprises a sequence that is not hybridized with the target sequence or with a sequence complementary to the target sequence. The sequence not hybridizing with, for example, a sequence X, refers to a sequence having low homology with the sequence X.

In the present invention, the term “monitoring nucleic acid probe” refers to a nucleic acid probe comprising a sequence complementary to the sequence comprised in the monitoring nucleic acid. In the case using a DNA microarray wherein the nucleic acid probe is immobilized on a substrate, the monitoring nucleic acid probe is immobilized on the same substrate. The monitoring nucleic acid probe forms a hybrid with the monitoring nucleic acid.

In the present invention, the term “sample solution” refers to a solution in which a target nucleic acid may exist. The sample solution is subjected to the detection method of the present invention.

In the present invention, the term “substrate” refers to a support on which a nucleic acid probe is immobilized. The substrate, together with the nucleic acid probe immobilized thereon, constitutes a device such as a DNA microarray.

Hereinafter, the embodiments of the present invention will be described in detail.

The monitoring nucleic acid probe used in the present invention shows signal intensity varying in the optimum temperature range for washing a target nucleic acid (optimum temperature range for washing) and in a temperature range in the vicinity of the optimum temperature range for washing.

The monitoring nucleic acid probe is immobilized on a substrate and hybridized with the monitoring nucleic acid. A current obtained from the hybrid thus formed is the signal mentioned above, and its intensity is measured.

Generally, washing is conducted after hybridization, thereby dissociating the hybrid to some extent. The signal intensity is thereby decreased. As the temperature of a wash is increased, the amount of the dissociated hybrid is increased. Accordingly, when the temperature of a wash is changed under the conditions where the salt concentration and pH are kept constant, the rate of reduction in signal intensity is changed. That is, the reduction in signal intensity is increased as the temperature of a wash is increased.

As will be described later, the optimum temperate range for washing is an allowable temperature range of a wash, which is used in washing a target nucleic acid in a washing step in the detection method of the present invention. First, a probe as a candidate for the monitoring nucleic acid probe is hybridized with its complementary chain and then washed at a temperature in the optimum temperature range for washing and in the vicinity of the range, and the signal intensity from the probe formed hybrid is measured. This measurement is conducted at a plurality of varying washing temperatures. Based on the measurement results, a probe whose signal intensity changes depending on the change in washing temperature is selected as the monitoring nucleic acid probe. Particularly, a probe whose signal intensity changes significantly in the vicinity of the border temperatures of the optimum temperature range for washing is selected.

The melting temperature (Tm) is liable to influence the change in signal intensity of a nucleic acid attributable to a change in washing temperature. A probe having a low melting temperature (Tm) changes its signal intensity when washed at low temperature. On the other hand, a probe having a high melting temperature (Tm) changes its signal intensity at high temperature. Therefore, the Tm value can be an indicator for selection.

The Tm value also varies depending on the content of GC in a nucleic acid. Generally speaking, however, a nucleic acid of shorter chain has a lower Tm value, while a nucleic acid of longer chain has a higher Tm value. The Tm value of a relatively short nucleic acid can be calculated for example by using a Wallace method. In the Wallace method, the Tm value is calculated assuming that the force of bonding between guanine and cytosine is 4° C. and the force of bonding between adenine and thymine is 2° C.

In a preferable embodiment of the present invention, a probe with a high rate of change in signal intensity in the above temperature range is selected as the monitoring nucleic acid probe. As the rate of change in signal intensity becomes higher, the difference in signal intensity resulting from a minute change in the washing level can be detected more accurately.

The efficiency of amplification of a nucleic acid, immobilization of a probe on a substrate in a process for producing a chip, hybridization and washing vary from operation to operation, and thus the total efficiency of the method of detecting a nucleic acid varies among detection tests. This is referred to as variation among detection tests. Generally, the variation among detection tests is calculated as the rate of variation by considering all factors included in steps of the detection test.

When the variation among detection tests is significant, it is difficult for a probe with a low rate of change in signal intensity to accurately identify an abnormality in the washing step. Hence, in a preferable embodiment of the present invention, a probe with a high rate of change in signal intensity is preferably used.

The rate of change in signal intensity can be calculated as follows:

First, the monitoring nucleic acid probe is hybridized with the monitoring nucleic acid. Without washing, the signal intensity obtained from hybrid generated above is measured. Its measured value is assumed to be 100. Then, a washing fluid having a constant salt concentration and constant pH is used in washing at varying temperatures, and then the signal intensity is measured. The signal intensities thus determined at the respective temperatures are relatively expressed as signal intensity ratio where the above measured value without washing is assumed to be 100.

As the washing temperature is gradually increased, the signal intensity is decreased gradually in an initial stage. However, at a certain point in time, the signal intensity is rapidly decreased. When the rate of change in this signal intensity is expressed in a graph as a function of signal intensity ratio against temperature, an approximate line can be drawn in the range where the signal intensity is significantly decreased. From the inclination of this approximate line, the rate of change in signal intensity can be calculated.

In one embodiment, the approximate line is prepared in the following manner. First, signal intensities determined at 4 points in the washing temperature range of ±2° C. from the washing temperature at which the signal intensity ratio reaches 50 are selected. Then, an approximate line is formed from the values at the 4 points.

In a preferable embodiment of the present invention, the monitoring nucleic acid probe with a rate of change of 13 or more (preferably 15 or more) in signal intensity is used when the variation among detection tests is less than 20%. In another preferable embodiment, the monitoring nucleic acid probe with a rate of change of 18 or more in signal intensity is used when the variation among detection tests is not less than 30%.

As the rate of change in signal intensity from the monitoring nucleic acid probe is increased, minutely different washing levels can be accurately detected regardless of the variation among detection tests.

For the monitoring nucleic acid probe selected as described above, the optimum signal intensity range (optimum signal intensity for washing) is determined. The optimum signal intensity range refers to the range of signal intensities obtained when washing was conducted in the optimum temperature range for washing. The optimum signal intensity range is determined on the basis of signal intensities obtained after washing at varying temperatures in the optimum temperature range for washing, under the conditions where the salt concentration and pH are kept, as described above. Particularly, the range is determined preferably on the basis of signal intensities in washing at the border temperatures of the above temperature range.

In one embodiment, the signal intensity obtained after washing at the upper-limit temperature of the optimum temperature range for washing is regarded as the lower limit of the optimum signal intensity range for washing. The signal intensity obtained after washing in the lower-limit temperature of the optimum temperature range for washing is regarded as the upper limit of the optimum signal intensity range for washing.

The monitoring nucleic acid probe should be a probe with a high rate of change in signal intensity at both the upper- and lower-limit temperatures of the optimum temperature range for washing. A single probe may be used as long as it can satisfy this condition. When there is no probe satisfying this condition, a set of at least two probes may be used, that is, a probe with a high rate of change in signal intensity at the upper-limit temperature of the above temperature range and a probe with a high rate of change in signal intensity at the lower-limit temperature of the above temperature range, may be simultaneously used. Alternatively, a plurality of probes with high rates of change in signal intensity at arbitrary temperatures in the above temperature range and in the vicinity thereof may be used.

In the present invention, the optimum temperature range for washing means the temperature at which an unspecific hybrid generated by hybridization between the target nucleic acid and the nucleic acid probe is selectively removed. Actually, the temperature range can be decided based on the temperature where the signal intensity from an unspecific hybrid and a specific hybrid are made different from each other after washing. Preferably, the temperature range is decided based on the temperature where both the intensities are made clearly different from each other, more preferably the temperature where the signal intensity from an unspecific hybrid is made almost the same as that of the negative control.

This temperature range varies depending on the target sequence to be discriminated and the structure and type of the target nucleic acid. Generally, as the similarity between the sequences (for example wild-type and mutant-type sequences) to be discriminated from each other is increased, an unspecific hybrid is generated more easily, and thus the optimum temperature range becomes narrower. When the object to be detected is a nucleic acid with one-base substitution, insertion or deletion, the optimum temperature range is the narrowest. By way of example, when the detection object is a nucleic acid with single nucleotide polymorphism, the optimum temperature range is experimentally about 2 to 6 degrees in width when under the conditions of a constant salt concentration and constant pH, only the temperature is changed. There are also nucleic acids whose optimum temperature range is 2 to 3 degrees in width. In such cases, in detection for example with an automatic inspection apparatus, the temperature of washing fluid should be strictly controlled within 1 to 1.5 degrees from the center of the optimum temperature range.

As described above, the optimum temperature range for washing varies depending on the target nucleic acid to be detected. Accordingly, the optimum temperature range for washing may be arbitrarily determined by those who carry out the method of detecting a nucleic acid according to the present invention.

In the present invention, the temperature in the vicinity of the optimum temperature range for washing refer to a temperature in and around the optimum temperature range, for example a temperature within ±3° from the upper- and lower-limit temperatures of the optimum temperature range for washing. The upper- and lower-limit temperatures of the temperature range are intended to encompass temperatures at and around the border temperatures of the temperature range.

Now, the monitoring nucleic acid used in the present invention will be described in detail. The nucleic acid may be the same nucleic acid as the target nucleic acid or may be a nucleic acid different from the target nucleic acid. The monitoring nucleic acid may be an artificially produced nucleic acid analog or may be a nucleic acid amplified as necessary from genomic DNA, genomic RNA or mRNA of an individual. A gene of a living thing completely different from the living thing from which the target nucleic acid was derived may be searched and used.

When the monitoring nucleic acid is the same nucleic acid as the target nucleic acid, the sequence of the monitoring nucleic acid is comprised in a region different from that of the target sequence. When the monitoring nuclei acid is a nucleic aid different from the target nucleic acid, both the nucleic acids may be prepared by simultaneous amplification in the same vessel. Alternatively, the respective nucleic acids may be separately prepared. In this case, the monitoring nucleic acid is added to a sample solution. The concentration of the nucleic acid added may be arbitrarily determined based on the concentration of the target nucleic acid. The saturated concentration of the target nucleic acid after amplification is approximately constant. The number and amount of the probes immobilized on a substrate are also approximately constant. Accordingly, the concentration of the nucleic acid added can also be approximately constant.

Now, the steps in the method of detecting a target nucleic acid according to the present invention will be described in detail.

First, a nucleic acid probe for target nucleic acid, which comprises a sequence complementary to a sequence of a target acid nucleic acid, a monitoring nucleic acid, and a monitoring nucleic acid probe are prepared. Then, the nucleic acid probe and the monitoring nucleic acid probe are immobilized on a substrate. For convenience sake, the substrate having the nucleic acid probes immobilized thereon is referred to herein as probe-immobilized substrate. The respective nucleic acids and the probe-immobilized substrate may be prepared in advance prior to the detection test.

Alternatively, a sample solution subjected to the nucleic acid detection test is prepared. The sample solution has the possibility of containing the target nucleic acid and contains the monitoring nucleic acid. The target nucleic acid and the monitoring nucleic acid may be those extracted from individuals. The individuals include, but are not limited to, humans, nonhuman animals, plants, viruses, and microorganisms such as microbes, bacteria, yeasts and mycoplasma. Their nucleic acids can be obtained from, for example, collected blood, serum, leukocyte, urine, feces, semen, saliva, tissue, biopsy sample, oral mucosa, cultured cell, sputum, and the like. The extraction method is not particularly limited, and commercially available nucleic acid extraction kits such as QIAamp (manufactured by QIAGEN) and Sumaitest (manufactured by Sumitomo Metal Industries, Ltd.), and the like may also be used.

The extracted nucleic acid is amplified as necessary by an amplification method known in the art. The method that can be used includes, for example, polymerase chain reaction (PCR), loop-mediated isothermal amplification (LAMP), isothermal and chimeric primer-initiated amplification of nucleic acids (ICAN), nucleic acid sequence-based amplification (NASBA), strand displacement amplification (SDA), ligase chain reaction (LCR), and rolling circle amplification (RCA). The resulting amplification product is fragmented as necessary or made single-stranded. A means of making the amplification product single-stranded includes, for example, heat denaturation, a method of using beads, an enzyme etc., and a method of transcription reaction with T7 DNA polymerase. When a single-stranded region exists in the product amplified by LAMP, ICAN or the like, and this single-stranded region is used as a target sequence, it can be subjected directly to hybridization (see, for example, JP-A 2005-143492 (KOKAI)).

Separately from the above preparation, the optimum signal intensity range for washing is determined for the monitoring nucleic acid probe. This determination may be conducted as described above in detail.

Then, the sample solution prepared as described above is subjected to hybridization on the probe-immobilized substrate. In this hybridization step, the target nucleic acid is hybridized with the nucleic acid probe for target nucleic acid, and the monitoring nucleic acid nucleic acid is hybridized with the monitoring nucleic acid probe, on the probe-immobilized substrate. Then, unspecific hybrids generated in the hybridization step are removed by washing.

After washing, the signal intensity from the nucleic acid probe and the signal intensity from the monitoring nucleic acid probe are measured respectively.

The method of the present invention further includes an examination step of judging whether the washing step was normally conducted or not. In this examination step, it is judged that when the signal intensity obtained from the monitoring nucleic acid probe in the measuring step is within the previously determined signal intensity range (optimum signal intensity range for washing), the washing step was normally conducted. On the other hand, when the obtained signal intensity is outside the signal intensity range, it is judged that there was abnormality in the washing step. Particularly, when the obtained signal intensity is higher than the upper limit of the signal intensity range, it is judged that there was an abnormality in the washing step.

In the conventional method of detecting a nucleic acid, a positive control whose signal intensity is not changed by a change in the washing temperature is used. Conventionally, it has been judged that when the signal intensity from the positive control is not lower than a predetermined value, the test was normally conducted. Accordingly, only the lower limit of the signal intensity is established without regarding too high signal intensity as problematic.

However, the nucleic acid detection method of the present invention, unlike the conventional method, uses a probe whose signal intensity is changed significantly by washing in the optimum temperature range for washing. The signal intensity from such a probe is not significantly changed at a temperature outside the optimum temperature range for washing. For example, when the washing temperature is lower than the optimum temperature range for washing, the signal intensity remains high. It can thereby be judged that when signal intensity higher than the predetermined signal intensity range is detected, the washing level is lower than established. On the other hand, it can be judged that when a signal intensity lower than the predetermined signal intensity range is detected, the washing level is higher than established.

The salt concentration used in the method of detecting a nucleic acid is generally lower in washing than in hybridization. It follows that when washing is not normally conducted in the step of washing after hybridization, for example when the washing step is conducted with a hybridization solution having a high salt concentration, an unspecific signal remains thus bringing about in an abnormal result. The monitoring nucleic acid probe of the present invention is selected based on the change in signal intensity against temperature, and can detect such an abnormality in the salt concentration.

<Nucleic Acid Probe>

The chain length of the nucleic acid probe or the monitoring nucleic acid probe is not particularly limited, but is preferably in the range of 5 to 50 bases, more preferably in the range of 10 to 40 bases, and still more preferably in the range of 15 to 35 bases.

The nucleic acid probe may be unmodified or may be modified with reactive functional groups such as an amino group, a carboxyl group, a hydroxyl group, a thiol group and a sulfonyl group, or substances such as avidin and biotin for immobilization onto a substrate. A spacer may be introduced between the functional group and the nucleotide. As the spacer, an alkane skeleton, an ethylene glycol skeleton, or the like may be used.

The substrate on which the nucleic acid probe is to be immobilized may be composed of materials including, but not limited to, nitrocellulose film, nylon film, microtiter plate, glass, silicon, electrode, magnet, beads, plastics, latex, synthetic resins, natural resins, and optical fiber.

<Probe-Immobilized Substrate>

As one example of the probe-immobilized substrate, a nucleic acid microarray is schematically shown in FIG. 1. The microarray in this example is provided with an immobilization region 2 on a substrate 1. A nucleic acid probe is immobilized on the immobilization region 2. Such a nucleic acid microarray can be produced by a method known in the art. The number and arrangement of the immobilization regions 2 on the substrate 1 can be appropriately designed and altered as necessary by those skilled in the art. One type or plural types of nucleic acid probes may be immobilized on one substrate, and the number and type of the probes may be arbitrarily selected. The nucleic acid microarray as shown in this example is preferably used in a method of detection with fluorescence.

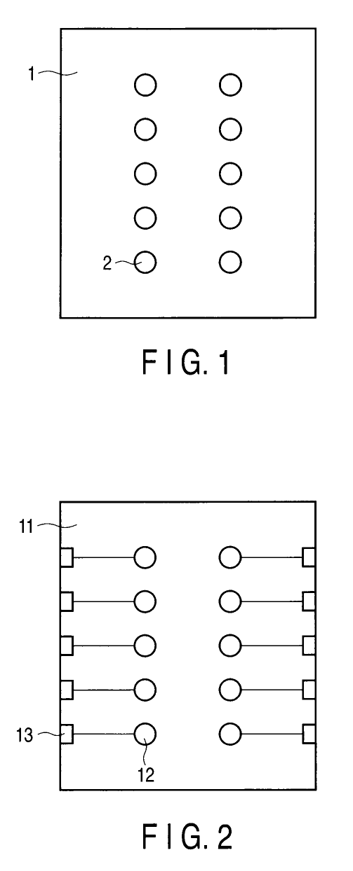

Another example of the probe-immobilized substrate is shown in FIG. 2. The nucleic acid microarray in FIG. 2 is provided with an electrode 12 on a substrate 11. A nucleic acid probe is immobilized on the electrode 12. The electrode 12 is connected to a pad 13. Electrical information from the electrode 12 is acquired via the pad 13. Such a nucleic acid microarray can be produced by a method known in the art. The number and arrangement of the electrodes 12 on the substrate 11 can be appropriately designed and altered as necessary by those skilled in the art. One type or plural types of the nucleic acid probe for target nucleic acids may be immobilized on one substrate, and the number and type of the probes may be arbitrarily selected. The nucleic acid microarray in this example may be provided as necessary with a reference electrode and a counter electrode.

The materials that can be used in the electrode include, but are not limited to, gold, a gold alloy, silver, platinum, mercury, nickel, palladium, silicon, germanium, gallium, and tungsten and alloys thereof, carbons such as graphite and glassy carbon, and oxides and compounds thereof.

The nucleic acid microarray as shown in this example is preferably used in a method of electrochemical detection.

<Hybridization Conditions>

Hybridization is carried out under suitable conditions where hybrids are sufficiently formed. The conditions are preferably those under which specific hybrids can be formed predominately over unspecific hybrids. The suitable conditions vary depending on the type and structure of the target nucleic acid, the type of bases contained in the target nucleic acid, and the type of the nucleic acid probe. For example, hybridization is conducted in a buffer with an ionic strength in the range of 0.01 to 5 and at pH in the range of 5 to 9. The reaction temperature may be in the range of 10 to 90° C. The reaction efficiency may be increased by stirring or shaking. The reaction solution may contain a hybridization promoter such as dextran sulfate, salmon sperm DNA or bovine thymus DNA, as well as EDTA or a surfactant.

<Washing Conditions>

The washing fluid used is preferably a buffer with an ionic strength in the range of 0.01 to 5 and at pH in the range of 5 to 9. The washing fluid preferably contains a salt and a surfactant. Examples of the washing fluid that can be preferably used include SSC solution, Tris-HCl solution, Tween 20 solution and SDS solution. The washing temperature is set in the optimum temperature range for washing as described above. The washing fluid is passed through, or retained on, the surface of the probe-immobilized substrate or on the region where the nucleic acid probe was immobilized. Alternatively, the probe-immobilized substrate may be immersed in the washing fluid. In this case, the washing fluid is preferably accommodated in a container capable of temperature control.

<Detection Method>

In detection of a hybrid formed in the hybridization step, a fluorescence detection system and an electrochemical detection system can be used.

(a) Fluorescence Detection System

The hybrid is detected with a fluorescence-labeled substance. Primers used in the step of amplifying the nucleic acid may be labeled with a fluorescently active substance such as a fluorescence dye such as FITC, Cy3, Cy5 or rhodamine. Alternatively, a second probe labeled with such a substance may be used. A plurality of labeling substances may also be simultaneously used. The label in the labeled sequence or in the second probe is detected with a detector. A suitable detector is used depending on the label used, for example, a fluorescence detector is used.

(b) Electrochemical Detection System

A double strand-recognizing substance known in the art is used. The double strand-recognizing substance may be selected from Hoechst 33258, Acridine Orange, quinacrine, daunomycin, a metallointercalator, a bisintercalator such as bisacridine, a trisintercalator and a polyintercalator. The double strand-recognizing substance may be modified with an electrochemically active metal complex, for example, ferrocene or viologen.

The concentration of the double strand-recognizing substance, though varying depending on its type, is used generally in the range of 1 ng/mL to 1 mg/mL. In this case, a buffer having an ionic strength ranging from 0.001 to 5 and a pH ranging from 5 to 10 is preferably used.

The measurement is performed by applying at least a potential at which a double strand-recognizing substance can react electrochemically, followed by measuring a reaction current derived from the double strand-recognizing substance. The potential may be applied by sweeping it at a constant rate or in a pulse fashion. Alternatively, a constant potential may be applied. The current and voltage may be controlled by a device such as a potentiostat, a digital multimeter or a function generator. The electrochemical measurement can be carried out according to methods known in the art. For example, a method described in JP-A 1998-146183 (KOKAI) can be used.

### EXAMPLES

Hereinafter, the present invention will be described in detail by reference to the Examples.

### Example 1

An example showing a method of determining the optimum temperature for washing a target nucleic acid will be described in detail. In this example, detection of a nucleic acid having single nucleotide polymorphism (SNP) was conducted. The target nucleic acid is a nucleic acid containing single nucleotide polymorphism G590A of NAT2 gene. The target nucleic acid was amplified by LAMP. (1) Primers

Synthetic DNA oligo primers used in amplification of the target nucleic acid are shown in Table 1.

TABLE 1

| | | |
|---|---|---|
| SEQ ID NO: | Primer name | Sequence |

| | | |
|---|---|---|
| 1 | F3 primer | CTGGGAAGGATCAGCCTC |
| 2 | FIP primer | GTTTGTAATATACTGCTCTCTCCTG- |
| | | CCTTGCATTTTCTGCTTGAC |
| 3 | B3 primer | AAATGAAGATGTTGGAGACG |
| 4 | BIP primer | CACCAAAAAATATACTTATTTACGC- |
| | | CTGCAGGTATGTATTCATAGACTC |
| 5 | LP primer | GTACCAGATTCCTCTCTCTTCT |

(2) LAMP Reaction Solution

The composition of a reaction solution used in LAMP is shown in Table 2.

TABLE 2

| | | |
|---|---|---|
| 2 × Buffer | 12.5 | μL |
| Tris•HCl pH 8.0 40 mM | | |
| KCl 20 mM | | |
| MgSO₄ 16 mM | | |
| (NH₄)₂SO₄ 20 mM | | |
| Tween20 0.2% | | |
| Betaine 1.6 M | | |
| dNTP 2.8 mM | | |
| F3 primer (10 μM) | 0.5 | μL |
| B3 primer (10 μM) | 0.5 | μL |
| FIP primer (20 μM) | 2 | μL |
| BIP primer (20 μM) | 2 | μL |
| LP primer (10 μM) | 1 | μL |
| Bst DNA Polymerase | 1 | μL |
| Human genome (30 ng/μL) | 1 | μL |
| Sterilized ultrapure water | 4.5 | μL |
| Total | 25 | μL |

(3) Nucleic Acid Amplification

As templates for amplification, three types of human genomes, that is, G-type, A-type, and G/A-type were used. Amplification was conducted at 63° C. for 1 hour. Thereafter, the enzyme was inactivated at 80 ° C. for 2 minutes. A negative control was prepared by adding sterilized water in place of the human genome. The reaction solution was subjected to agarose gel electrophoresis. As a result, a ladder-shaped pattern characteristic of the LAMP product appeared, and the amplification was thus confirmed. In the reaction solution of the negative control, no amplification was confirmed.

(4) Nucleic Acid Probes

Three types of nucleic acid probes used in this example are shown in Table 3.

TABLE 3

| SEQ | | |
|---|---|---|
| ID NO: | Probe name | Sequence |
| 6 | Negative control | GTGCTGCAGGTGCG |
| 7 | 590 G-type | TTGAACCTCGAACAATTGAAGATTTT |
| 8 | 590 A-type | TTGAACCTCAAACAATTGAAGATTTTG |

The nucleic acid probe for negative control has a sequence irrelevant to the sequence of NAT2 gene. In Table 3, probe 590G is a nucleic acid probe for detection of the wild-type nucleic acid. Probe 590A is a probe for detection of the mutant-type nucleic acid. These 3 probes were modified at the 3′-terminals thereof with thiol for immobilization on an electrode.

(5) Preparation of a Microarray

A substrate provided with gold electrodes was used. Each nucleic acid probe was immobilized on a gold electrode by utilizing the strong chemical bonding between thiol and gold. First, a solution containing the probes modified with thiol at the terminals thereof as described above was spotted on the gold electrode and left at 25° C. for 1 hour. Thereafter, the substrate was washed with 0.2×SSC solution. Then, the substrate was washed with ultrapure water and then air-dried. The same probe was immobilized on 4 electrodes. The prepared microarray was set in a special cassette. This cassette is provided with a flow path through which a solution flows on only the nucleic acid probe-immobilized site.

(6) Hybridization

2×SSC salt was added to the LAMP product-containing reaction solution obtained in (3) above. This solution was injected into the microarray cassette prepared in (5) above. Then, the cassette was set in a nucleic acid automatic inspection apparatus (see Rinsho Byori. 55 216-223, 2007). The hybridization, washing and detection steps were conducted in the automatic inspection apparatus. Hybridization was conducted at 55° C. for 20 minutes. Washing was conducted by sending 0.2×SSC solution set in the inspection apparatus to the cassette and leaving it at 44 to 52° C. for 20 minutes. In detection, a phosphate buffer containing 50 μM Hoechst 33258, also set in the apparatus, was sent to the cassette and retained for 10 minutes. Thereafter, the oxidation current response of Hoechst 33258 was detected. The experiment was conducted by washing at 9 washing temperatures changed by 1° C. in the washing temperature range described above.

(7) Results

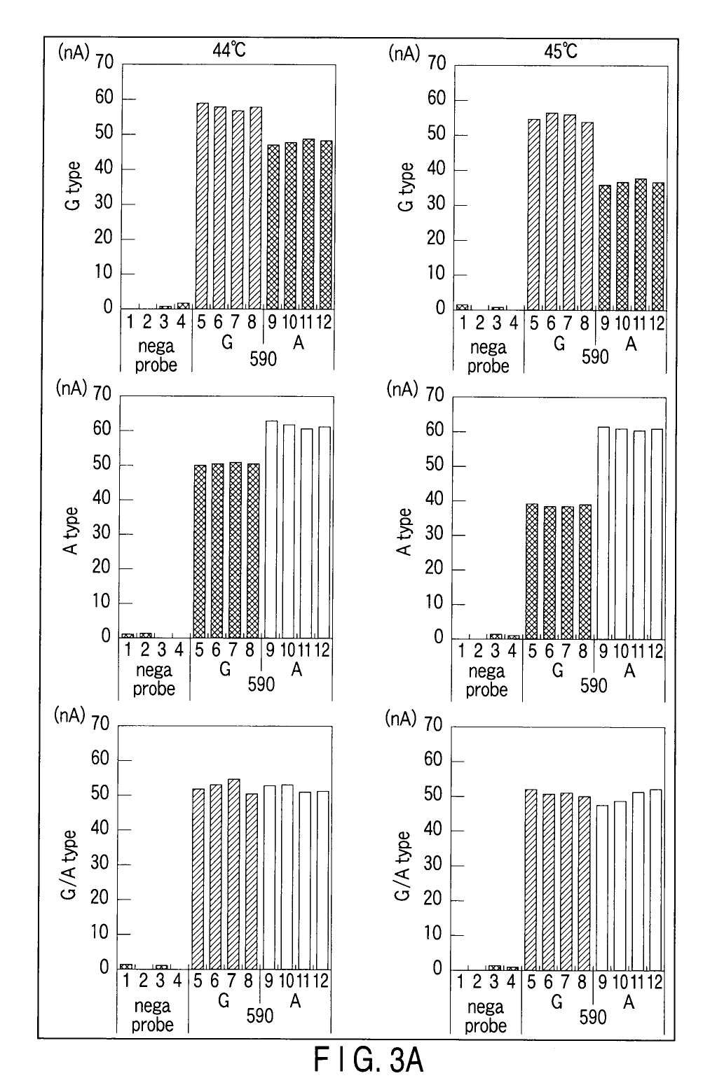

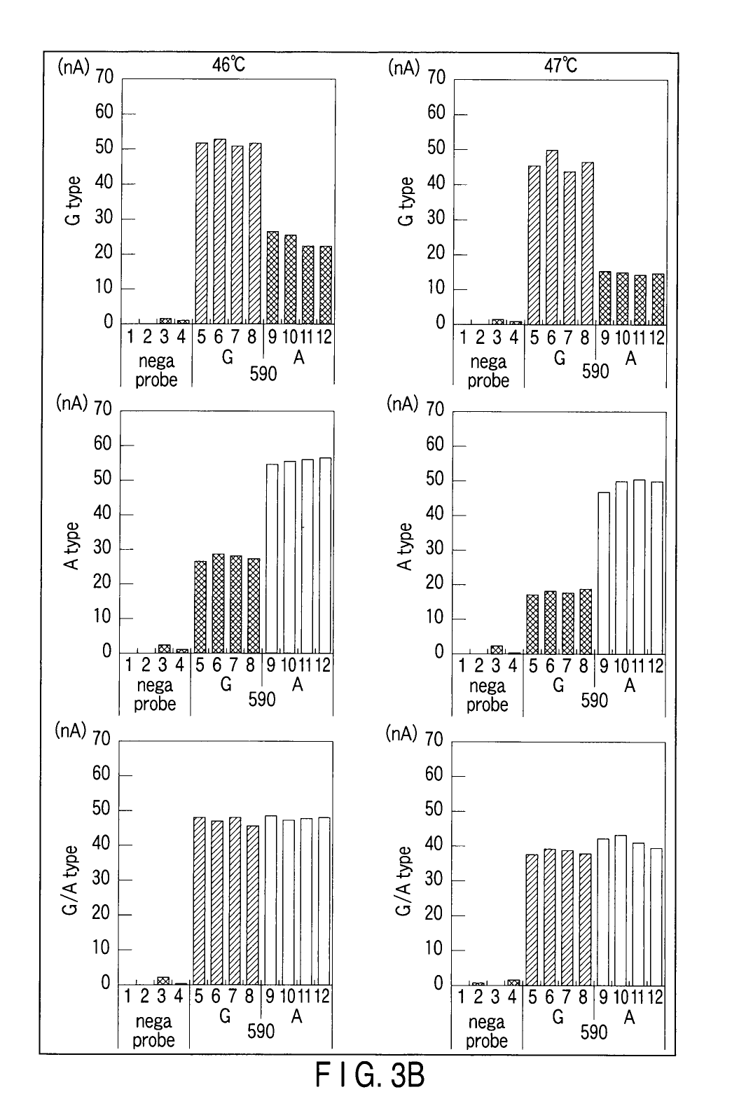

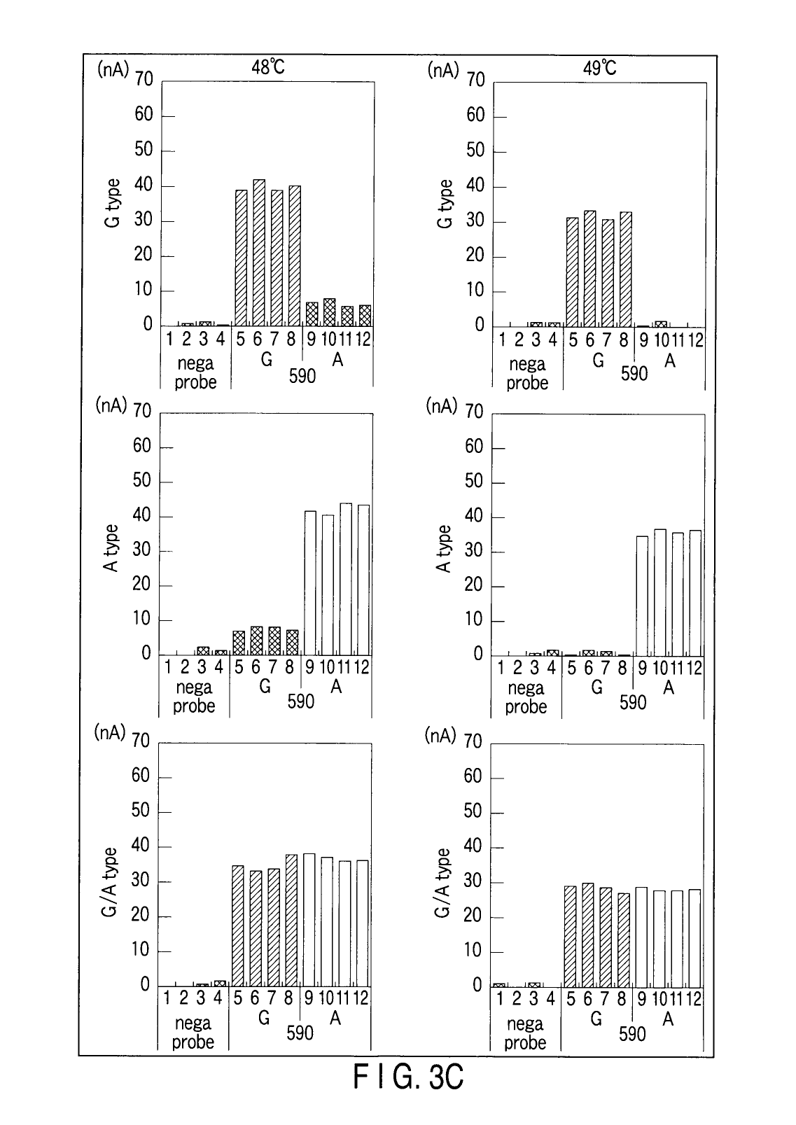

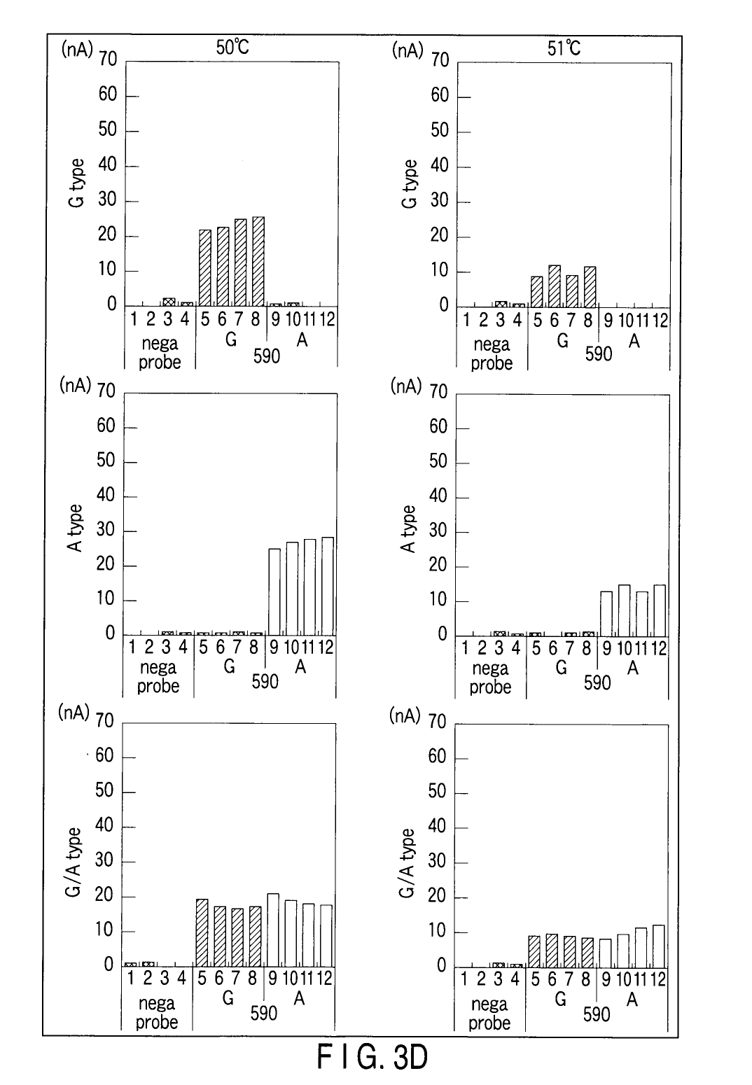

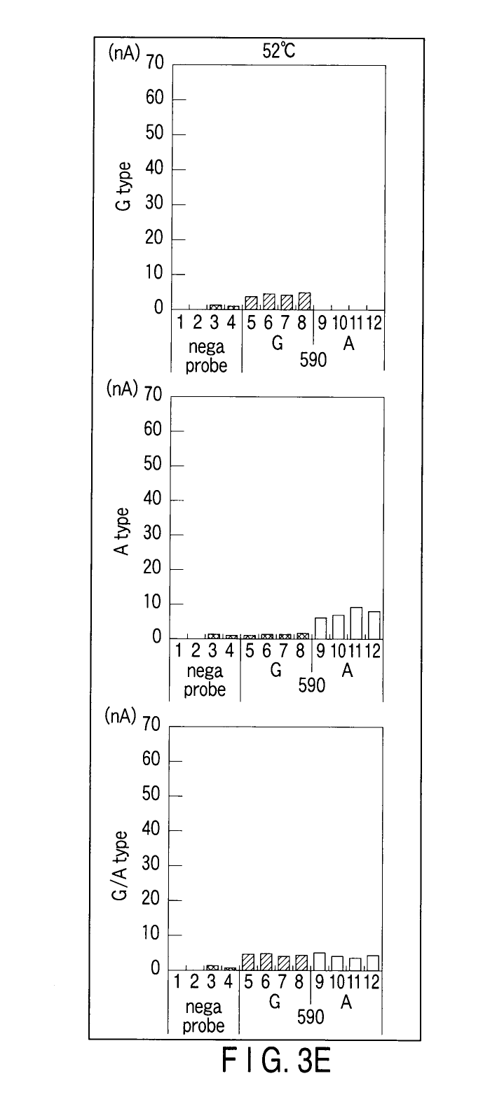

The results are shown in FIGS. 3A to 3E. In FIGS. 3A to 3E, as the washing temperature was decreased from 46 to 45° C., the G-type target nucleic acid showed a strong signal from the unspecific A-type probe, and the A-type target nucleic acid showed an increasing signal from the unspecific G-type probe. When the washing temperature was 44° C., the unspecific signal was further increased so that the G- and A-type homogeneous ones were hardly discriminated from the heterogeneous ones. When the washing temperature was not lower than 51° C., the specific signal was reduced.

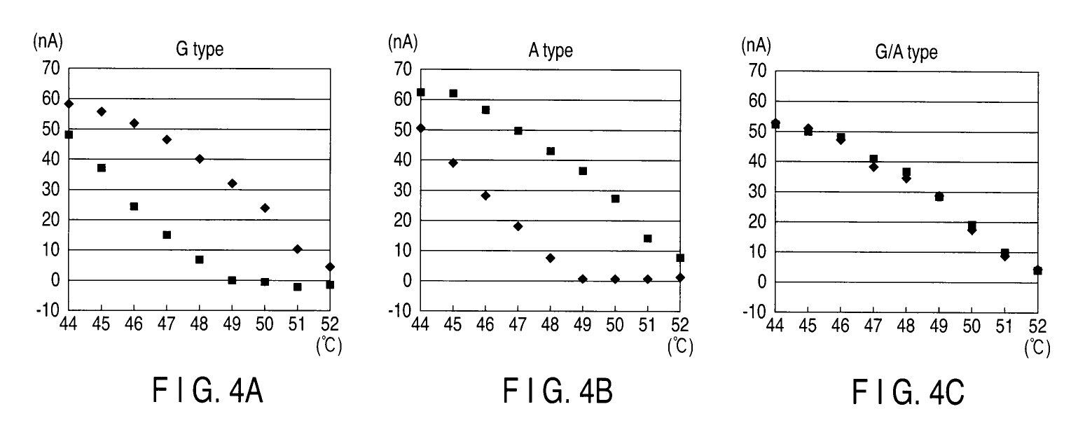

The average value of 4 electrodes in FIGS. 3A to 3E was calculated and plotted on a graph shown in FIGS. 4A to 4C.

In FIGS. 4A to 4C, the rhombic mark shows the signal from the G-type probe, and the square mark shows the signal from the A-type probe. From FIGS. 3 and 4, it was revealed that the optimum washing temperature at which the A-type, G-type and heterogeneous ones can be clearly identified is 47 to 50° C.

### Example 2

An example showing a method of selecting the monitoring nucleic acid probe will be described in detail. Five kinds of nucleic acid probes of different chain lengths were used to examine the relationship between washing temperature and signal intensity. Using the synthetic DNA oligo primers shown in Table 4, LAMP was carried out with the human genome as the template. The LAMP reaction solution and amplification conditions are the same as in Example 1.

TABLE 4

| | | |
|---|---|---|
| SEQ ID NO: | primer name | Sequence |

| | | |
|---|---|---|
| 9 | F3 primer | GAGCTTGGCATATTGTATCTATACC |
| 10 | FIP primer | TCACTTTCCATAAAAGCAAGGTTTTT- |
| | | AAGTAACTCTTAGATATGCAATAATT- |
| | | TTCCCAC |
| 11 | B3 primer | CTAGTCAATGAATCACAAATACGC |
| 12 | BIP primer | AGAAAGTAAAAGAACACCAAGAATCG- |
| | | ATGTAACATTTTACCTTCTCCATTTT- |
| | | GA |
| 13 | LP primer | CATCAACAACCCTCGGGAC |

The 5 kinds of nucleic acid probes of different chain lengths used in this example are shown in Table 5. The 5 kinds of probes have a sequence complementary fully to sequence in the LAMP product. The negative control used was the same probe as in Example 1.

TABLE 5

| | | |
|---|---|---|
| SEQ ID NO: | Probe length | Sequence |

| | | |
|---|---|---|
| 14 | 17 mer | GGGTTCCTGGGAAATAA |
| 15 | 21 mer | TATGGGTTCCTGGGAAATAAT |
| 16 | 23 mer | TTATGGGTTCCTGGGAAATAATC |
| 17 | 24 mer | TTATGGGTTCCTGGGAAATAATCA |
| 18 | 30 mer | TTGTTATGGGTTCCTGGGAAATAATC |
| | | AATG |

These probes were used to produce a microarray by the same method as in Example 1.

The LAMP product-containing reaction solution obtained above was subjected to hybridization on the microarray, and washed at a varying temperature in the same manner as in Example 1.

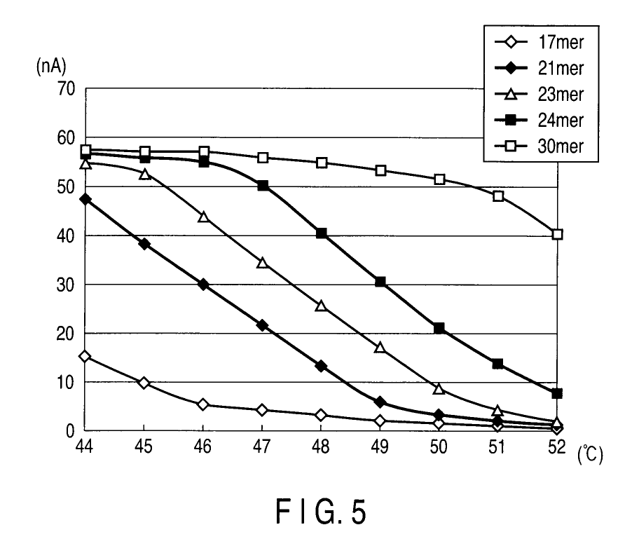

The results are shown in FIG. 5. The optimum temperature range for washing, determined in Example 1, is 47 to 50° C. In this temperature range, the 17-mer probe (SEQ ID NO: 14) showed low signal intensity. The change in the signal intensity by temperature was also low. In the above temperature range, the 30-mer probe (SEQ ID NO: 18) showed a high increase in signal, and the signal hardly changed. The 21-mer probe (SEQ ID NO: 15), 23-mer probe (SEQ ID NO: 16) and 24-mer probe (SEQ ID NO: 17) showed a drastic change in signal intensity in the above temperature range. Accordingly, these 3 probes can be used as the monitoring nucleic acid probes.

In the result in Example 1, the homogeneous and heterogeneous ones cannot be discriminated by washing at 44° C. On the other hand, a signal is hardly detected by washing at 52° C. From this result, for example by using the 23-mer probe (SEQ ID NO: 16), when the signal intensity from the probe was in the range of 5 to 40 nA, it can be assured that washing was conducted in the optimum temperature range. On the other hand, when the signal intensity from the probe is 40 nA or more, it can be judged that washing was conducted at a temperature lower than the above temperature range. It can also be judged that when the signal intensity is 5 nA or less, washing was conducted at a temperature higher than the above temperature range.

When the 21-mer probe (SEQ ID NO: 15) and 24-mer probe (SEQ ID NO: 17) are simultaneously used, they can be used as the monitoring nucleic acid probes. In this case, the signal intensity from the 21-mer probe is less than 25 nA and simultaneously the signal intensity from the 24-mer probe is 15 nA or more, it can be assured that washing was conducted at a temperature within the above temperature range. On the other hand, when the signal intensity from the 21-mer probe is 25 nA or more, it can be judged that washing was conducted at a temperature lower than the above temperature range. When the signal intensity from the 24-mer probe is less than 15 nA, it can be judged that washing was conducted at a temperature higher than the above temperature range.

### Example 3

The relationship between the rate of change in signal intensity and the variation among detection tests was examined. Four kinds of nucleic acid probes were used to determine the rate of change in signal intensity in the optimum temperature range for washing. Using the synthetic DNA oligo primers shown in Tables 4 and 6, LAMP was carried out with the human genome as the template. The LAMP reaction solution and amplification conditions are the same as in Example 1.

TABLE 6

| | | | Detection nucleic |
|---|---|---|---|
| SEQ ID NO: | Primer name | Sequence | acid probe |
| 19 | F3 primer | GTCTCCTGCCCTGACAGC | SEQ ID NO: 34 |
| 20 | FIP primer | CAGTGGTTTCTTCATCCCG- | |
| | | CAGGCACATCTTOTTCCCTC | |
| 21 | B3 primer | ACTCCTTGGTGTGGTCCTC | |
| 22 | BIP primer | GGAAGGCTCAGTATAAATAGCA- | |
| | | GTGCTGTAGCTGAGCTGCGG | |
| 23 | LB primer | GTCATTTATCCCAGTTGTGCAACC | |
| 24 | F3 primer | GAGGCTATTTTTGATCACATTGTA | SEQ ID NO: 35 |
| 25 | FIB primer | GAAAACCGATTGTGGTCAGAG | |
| | | GGTGTCTCCAGGTCAATCAA | |
| 26 | B3 primer | GGCTGCCACATCTGGGAG | |
| 27 | BIP primer | CATGGTTCACCTTCTCCTG- | |
| | | AGCTTCCAGACCCAGCAT | |
| 28 | LB primer | CCCAGTACAGAAGTTG | |
| 29 | F3 primer | GTGGGCTTCATCCTCAC | SEQ ID NO: 36 |
| 30 | FIP primer | AGCACTTCTTCAACCTCTTCCTG- | |
| | | TAAAGACAATACAGATCTGGTCG | |
| 31 | B3 primer | TGATAATTAGTGAGTTGGGTGAT | |
| | | GGGGAGAAATCTCGTGCCCA- | |
| 32 | BIB primer | AGGGTTTATTTTGTTCCTTATTC | |
| 33 | LB primer | AGTGAGAGTTTTAAACTCGAGC | |

The nucleic acid probes used in this example are the probe of SEQ ID NO: 16 shown in Table 5 and 3 kinds of probes shown in Table 7. The 3 kinds of probes are probes complementary fully to sequences in the LAMP product, respectively. The negative control used was the same probe as in Example 1.

TABLE 7

| | |
|---|---|
| SEQ ID NO: | Sequence |

| | |
|---|---|
| 34 | CCACCGTTCCCTGGCAG |
| 35 | AGGTGACCACTGACGGC |
| 36 | CCTGGTGATGGATCCCTTACTAT |

These probes were used to produce a microarray by the same method as in Example 1.

The LAMP product-containing reaction solution obtained above was subjected to hybridization on the microarray in the same manner as in Example 1.

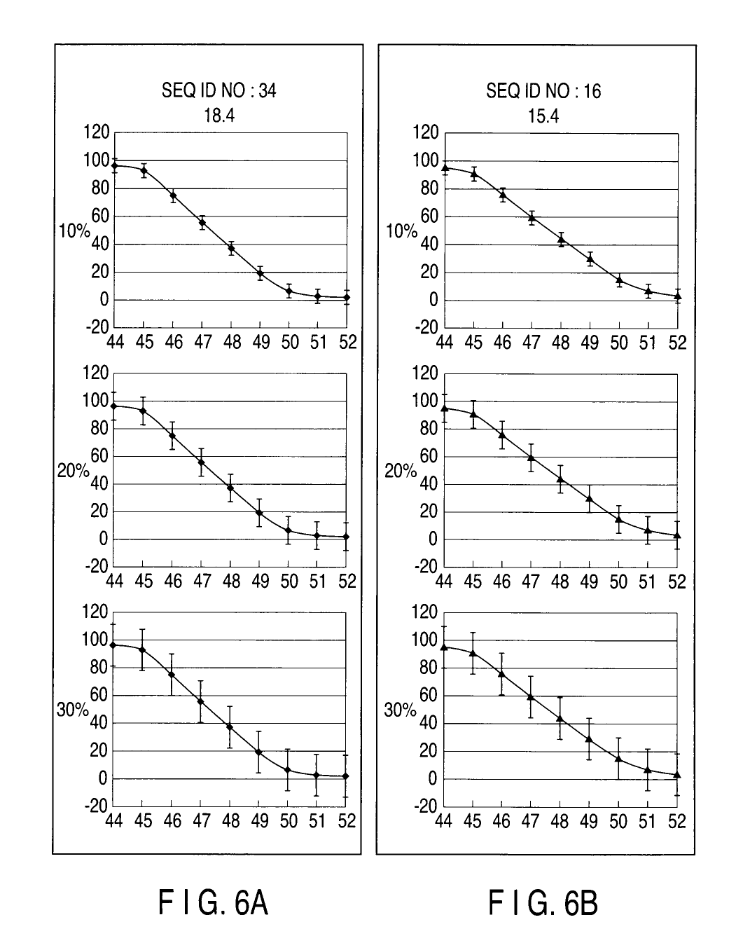

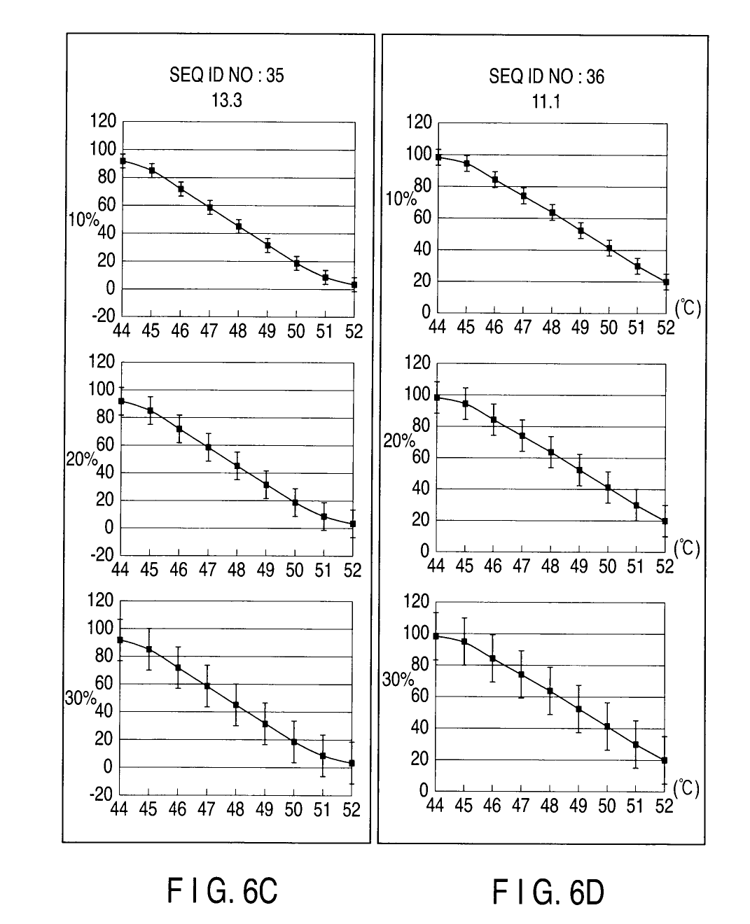

The results are shown in FIGS. 6A to 6D. An approximate line was drawn from 4 points within ±2° C. from the washing temperature at which the signal intensity ratio reached 50, thereby determining the rate of change in signal intensity.

The rates of change, in signal intensity, of the probes of SEQ ID NOS: 34, 16, 35 and 36 were 18.4, 15.4, 13.3 and 11.1, respectively. The error ranges where the variations among detection tests were 10%, 20% and 30% are shown in the graphs.

When the variation among detection tests is 10%, the difference of measured values, by a difference of 1° C., of the probe of SEQ ID NO: 36 with a rate of change of 11.1 in signal intensity is in the error range, thus making discrimination of the difference of 1° C. difficult. However, it is evident from the graph that as the rate of change in signal intensity is increased to 13.3, 15.4, and 18.4, the difference of measured values by a difference of 1° C. can be clearly discriminated.

Similarly, when the variation among detection tests is 20%, the difference of measured values, by a difference of 2° C., of the probe of SEQ ID NO: 36, is in the error range, thus making discrimination of the difference of 2° C. difficult. However, it is evident from the graph that as the rate of change in signal intensity is increased to 13.3, 15.4, and 18.4, the difference of 2° C. can be clearly discriminated.

When the variation among detection tests is 30%, it is difficult for the probe with a rate of change of 13.3 or 15.4 in signal intensity to discriminate the difference of 2° C. However, it is evident from the graph that the probe of SEQ ID NO: 34 with a rate of change of 18.4 in signal intensity can clearly discriminate the difference of 2° C.

From the foregoing, it was revealed that an abnormality in the washing step can be detected by using a probe with a rate of change of 13 or more in signal intensity when the variation among detection tests is less than 20%, or by using a probe with a rate of change of 18 or more in signal intensity when the variation among detection tests is 20% or more. For example, by using such probe in this example, the case where washing was conducted at the lower-limit temperature of 47° C. in the temperature range determined in Example 1 and the case where washing was conducted at a temperature lower than 45° C. can be discriminated.

In this example, a probe with a rapid rate of change in signal intensity at a washing temperature in the vicinity of 45 to 50° C. was used. However, those skilled in the art would appreciate that the optimum washing temperature varies according to the target nucleic acid or a difference in the detection conditions. It is easy for those skilled in the art select and use a monitoring nucleic acid probe having a suitable Tm value.

### Example 4

Detection of a target nucleic acid was conducted under abnormal washing conditions. Inconveniences assumed in washing conditions were that when the test was conducted with an automatic inspection apparatus, the washing temperature did not reach the optimal temperature, and that the washing fluid was not normally sent. The target nucleic acid used was the G-type LAMP product of NAT2 G590A in Example 1. As the monitoring nucleic acid probe, the 23-mer probe (SEQ ID NO: 16) in Example 2 was used.

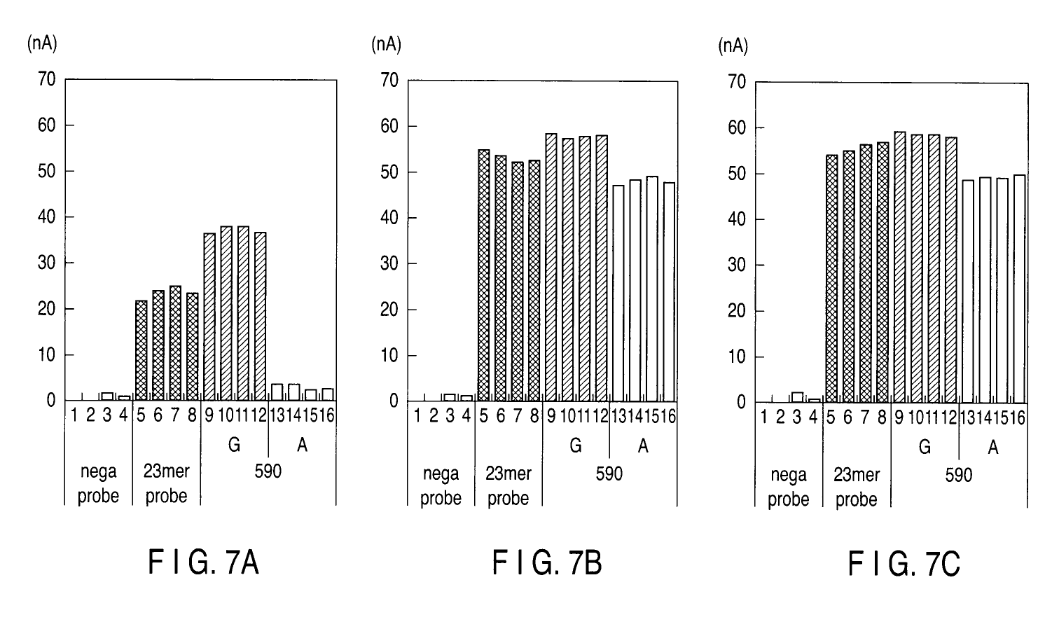

The results are shown in FIGS. 7A to 7C. FIG. 7A shows the result of detection wherein the washing temperature was 48.5° C. and the washing fluid was normally sent. FIG. 7B shows the result of detection wherein the washing temperature was 44° C. and the washing fluid was normally sent. FIG. 7C shows the result of detection wherein the washing temperature was 48.5° C. and the washing fluid was not sent normally.

When the washing fluid was normally sent, the solution having salt concentration of 0.2×SSC is used in washing. On the other hand, when the washing fluid was not normally sent, the solution having salt concentration of 2×SSC was used in washing, which is the salt concentration of the reaction solution for hybridization.

In FIG. 7A, the signal intensity from the monitoring nucleic acid probe was about 24 nA. This signal intensity was in the signal intensity range of 5 to 40 nA determined in Example 2. A strong signal was detected from the G-type probe. A signal was hardly detected from the A-type probe. From these results, it was revealed that a specific hybrid was formed, while an unspecific hybrid was hardly formed. Accordingly, these results can be said to be ideal detection results.

In FIGS. 7B and 7C, on the other hand, the signal intensity from the monitoring nucleic acid probe was about 55 nA. This intensity is outside the signal intensity range of 5 to 40 nA determined above. Both the results in FIGS. 7B and 7C indicate that the signal from the A-type probe is strong and an unspecific hybrid exists. The detection result in FIGS. 7B and 7C is hardly discriminated from the result of detection of heterogeneous ones, thus leading to a high possibility of erroneous judgment.

From the foregoing, it was revealed that when there is an abnormality in the washing step, the signal intensity from the monitoring nucleic acid probe comes to be outside the optimum signal intensity range for washing. Accordingly, whether the washing step was normally conducted or not can be judged by measuring signal intensity with a suitable monitoring nucleic acid probe. The erroneous judgment caused by an abnormality in the washing step can thereby be eliminated, and the accuracy of detection can be improved.

As described above, the monitoring nucleic acid probe is used according to the present invention, whereby an abnormality in the washing step can be detected, and the erroneous judgment resulting from an abnormality in the washing step can be avoided. Accordingly, the accuracy of detection of a target nucleic acid from among similar sequences can be improved.

Additional advantages and modifications will readily occur to those skilled in the art. Therefore, the invention in its broader aspects is not limited to the specific details and representative embodiments shown and described herein. Accordingly, various modifications may be made without departing from the spirit or scope of the general inventive concept as defined by the appended claims and their equivalents.

What is claimed is:

1. A method of detecting a target nucleic acid, comprising:
preparing the target nucleic acid and a monitoring nucleic acid for monitoring a washing level, the target nucleic acid comprises a target sequence and the monitoring nucleic acid comprises a sequence not to be hybridized with the target sequence or with a sequence complementary to the target sequence,
providing the target nucleic acid and the monitoring nucleic acid to a nucleic acid probe comprising a sequence complementary to the target sequence and a monitoring nucleic acid probe to monitor the washing level, the monitoring nucleic acid probe comprises a sequence complementary to a sequence comprised in the monitoring nucleic acid, thereby hybridizing the target nucleic acid with the nucleic acid probe and hybridizing the monitoring nucleic acid with the monitoring nucleic acid probe,
washing hybrids generated in the above step to remove unspecific hybrids,
measuring the signal intensity from the nucleic acid probe hybridized with the target nucleic acid and the signal intensity from the monitoring nucleic acid probe hybridized with the monitoring nucleic acid, respectively, and
examining whether the washing step has been normally conducted or not;
wherein the monitoring nucleic acid probe shows a change in signal intensity after hybridization with the monitoring nucleic acid and subsequent washing at a washing temperature changed in the optimum temperature range for washing and a vicinity of the range,
an optimum signal intensity range for washing having the upper and lower limits is determined in advance based on the signal intensity obtained from the monitoring nucleic acid probe which has been hybridized with monitoring nucleic acid and subsequent washed in the optimum temperature range for washing, and
the examining step comprises judging that when the signal intensity obtained from the monitoring nucleic acid probe hybridized with the monitoring nucleic acid is within the optimum signal intensity range for washing, the washing has been normally conducted, and that when the signal intensity is outside the optimum signal intensity range for washing, there has been an abnormality in the washing step.

2. The method according to claim 1, wherein when the signal intensity obtained from the monitoring nucleic acid probe hybridized with the monitoring nucleic acid is higher than the upper limit of the optimum signal intensity range for washing, it is judged that there has been an abnormality in the washing step.

3. The method according to claim 1, wherein when the signal intensity obtained from the monitoring nucleic acid probe without washing after hybridization with the monitoring nucleic acid is assumed to be 100, the monitoring nucleic acid probe shows a rate of change of 13 or more in signal intensity every 1° C. in washing at a varying washing temperature under the conditions of a constant salt concentration and constant pH.

4. The method according to claim 3, wherein the rate of change in signal intensity is the inclination of an approximate line in a function of signal intensity ratio against washing temperature in washing under the conditions of a constant salt concentration and constant pH.

5. The method according to claim 4, wherein the approximate line in a function of signal intensity ratio against washing temperature is a line formed from signal intensity at 4 points within ±2° C. from the washing temperature at which the signal intensity ratio reaches 50.

6. The method according to claim 1, wherein when the signal intensity obtained from the monitoring nucleic acid probe without washing after hybridization with the monitoring nucleic acid is assumed to be 100, the monitoring nucleic acid probe shows a rate of change of 15 or more in signal intensity every 1° C. in washing at a varying washing temperature under the conditions of a constant salt concentration and constant pH.

7. The method according to claim 6, wherein the rate of change in signal intensity is the inclination of an approximate line in a function of signal intensity ratio against washing temperature in washing under the conditions of a constant salt concentration and constant pH.

8. The method according to claim 7, wherein the approximate line in a function of signal intensity ratio against washing temperature is a line formed from signal intensity at 4 points within ±2° C. from the washing temperature at which the signal intensity ratio reaches 50.

9. The method according to claim 1, wherein when the signal intensity obtained from the monitoring nucleic acid probe without washing after hybridization with the monitoring nucleic acid is assumed to be 100, the monitoring nucleic acid probe shows a rate of change of 18 or more in signal intensity every 1° C. in washing at a varying washing temperature under the conditions of a constant salt concentration and constant pH.

10. The method according to claim 9, wherein the rate of change in signal intensity is the inclination of an approximate line in a function of signal intensity ratio against washing temperature in washing under the conditions of a constant salt concentration and constant pH.

11. The method according to claim 10, wherein the approximate line in a function of signal intensity ratio against washing temperature is a line formed from 4 points within ±2° C. from the washing temperature at which the signal intensity ratio reaches 50.

12. The method according to claim 1, wherein the optimum temperature range for washing is decided based on the temperature where the signal intensity from an unspecific hybrid and signal intensity from a specific hybrid are made different form each other after washing.

13. The method according to claim 1, wherein the monitoring nucleic acid is the same nucleic acid as the target nucleic acid, and a sequence used as the monitoring nucleic acid is different from the target sequence.

14. The method according to claim 1, wherein the monitoring nucleic acid is a nucleic acid different from the target nucleic acid.

15. The method according to claim 1, wherein the monitoring nucleic acid probe shows a change in signal intensity after washing in the temperature range between the upper and lower limits of the optimum temperature range for washing.

16. The method according to claim 1, wherein the monitoring nucleic acid probe is a set of at least 2 probes, one probe shows a change in signal intensity by washing at a temperature in the upper limit of, and in the vicinity of, the optimum temperature range for washing and the other probe shows a significant change in signal intensity by washing at a temperature in the lower limit of, and in the vicinity of, the optimum temperature range for washing.

17. The method according to claim 1, wherein the optimum signal intensity range for washing is determined on the basis of the signal intensity from the monitoring nucleic acid probe after hybridization with the monitoring nucleic acid and subsequent washing at a washing temperature changed in the optimum temperature range for washing under the conditions of a constant salt concentration and constant pH.

18. The method according to claim 17, wherein the signal intensity obtained after washing at the upper limit of the optimum temperature range for washing is the lower limit of the optimum signal intensity range for washing, and the signal intensity obtained after washing at the lower limit of the optimum temperature range for washing is the upper limit of the optimum signal intensity range for washing.
